(19) United States
(12) Patent Application Publication
(10) Pub. No.: US 2006/0262272 A1
(43) Pub. Date: Nov. 23, 2006

(54) Apparatus and method for application of tinted light and concurrent assessment of performance

(75) Inventors: John Douglas Anderson, Cambridge (GB); Ian Jordan, Ely (GB); Graham Stewart Brandon Street, Reading (GB)

Correspondence Address: BIRCH STEWART KOLASCH & BIRCH, PO BOX 747, FALLS CHURCH, VA, 22040-0747, US

(73) Assignee: Orthoscopics Limited, Reading (GB)

(21) Appl. No.: 11/271,828

(22) Filed: Nov. 14, 2005

Related U.S. Application Data
(63) Continuation-in-part of application No. 10/464,491, filed on Jun. 19, 2003.
(63) Continuation-in-part of application No. 10/946,619, filed on Sep. 22, 2004.
Application No. 10/946,619 is a continuation-in-part of application No. PCT/GB03/01362, filed on Mar. 28, 2003.
(63) Continuation-in-part of application No. PCT/GB04/02032, filed on May 12, 2004.

(30) Foreign Application Priority Data
Dec. 21, 2000 (GB) 0031384.1
Nov. 30, 2001 (GB) 0128705.1
Mar. 28, 2002 (GB) 0207303.9

(51) Int. Cl. A61B 3/10

(52) U.S. Cl. 351/221

(57) ABSTRACT

Apparatus and a corresponding method for the assessment of the effects of tinted illumination on a subject's visual system is provided. Colour controllable light sources provide the illuminant for an object field comprising features viewed against a background. A practitioner or the subject can effect movement of a feature such as a symbol or a ball. The subject views the feature at different locations and/or whilst in motion, having selected a tint for evaluation of visual comfort or performance. Some embodiments of the invention may be used to assess the effects of optimising tint for subjects who suffer from visual dyslexia or other impairments of the visual system. Other embodiments are directly operable by the subject in order to help the subject to select tinted eyewear for the enhancement of his or her visual performance.

This application is a continuation in part of application Ser. No. 10/464,491 filed on Jun. 19, 2003 (which is itself a continuation in part of PCT/GB01/005544 filed Dec. 17, 2004) and Ser. No. 10/946,619 filed Sep. 22, 2004 (which is itself a continuation in part of PCT/GB03/01362 filed Mar. 28, 2003) and a continuation of International Application No. PCT/GB2004/002032 filed on May 12, 2004 and for which priority is claimed under 35 USC §120. In addition, Applicants claim that this continuation in part application also claims priority of Application Nos. GB0031384.1 filed Dec. 21, 2000; GB0128705.1 filed Nov. 30, 2001; GB0207303.9 filed Mar. 28, 2002 under 35 USC §119. The entire contents of each of the above-identified applications are hereby incorporated by reference.

### BACKGROUND

The current invention is concerned with the provision of the illumination for a given task, and assessing the associated level of improvement for the subject undertaking said task. It may be used by a subject to select a preferred tint in order to enhance his or her visual comfort or performance.

It is known that the response of the visual system is affected by the stimuli, which it receives. The threshold for such stimulation varies between individuals and, under adverse conditions, can significantly reduce performance. When the visual system is over stimulated, it reacts in a number of ways. Amongst a variety of undesirable effects, which can be caused, two examples include a drop in convergence sufficiency and a reduction in the ability to accommodate or fuse images. It is apparent that for some it is necessary to modify the visual stimulus by changing the spectral distribution in a specific task e.g. reading and writing in school. In summary, it is well established that the colour of ambient lighting has a major influence on the effects of disorders such as dyslexia, epilepsy and migraine.

In U.S. Pat. No. 5,855,428 (Wilkins) apparatus is described in which the spectral distribution of light from a fluorescent lamp to illuminate a surface to support reading material is altered by the interposition of specifically selected broadband filters. By adjustment of the position of the selected filter or filters different colours and saturation thereof can be selected.

In US Patent Application No 2001/0005319 A1 (Ohishi et al.) an illumination control system, for general use, is described, in which the coordinates in colour space of the controlled illumination are arranged to follow a predetermined locus of points by mixing specific amounts of light from a plurality of differently coloured light emitting diodes (LED's).

Neither of these documents identifies the benefit of using sources which are characterised by providing light with a spectral distribution which is relatively narrow for application to the alleviation of symptoms. This would be the case for laser sources, super-luminescent LED's and conventional coloured LED's, which provide light with a typical spectral bandwidth of between 17 nm to around 50 nm. The provision of illumination, using additive light sources, such as LED's, for the quantitative diagnosis and alleviation of symptoms presented by or improving the comfort of an individual, is the subject of this invention.

Apparatus for the assessment of a subject's performance with and without prescription tinted spectacles, in which, inter alia, convergence, visual stability and perceived image size are tested under a variety of standard illuminants (such as daylight, fluorescent lighting and tungsten illumination), is described in U.S. patent application Ser. No. 10/946,619, incorporated by reference herein. This apparatus consists of an enclosure, illuminated internally with appropriate light sources, each of which is typically selected, as required. At the front, there is a viewing port to allow a subject to gaze into the enclosure. Inside, there is a motorised carriage, which allows a practitioner to move a target for viewing by the subject. The target, for example, might comprise a black dot on a white background. The illumination is selected and the subject's performance is tested with and without the prescribed tinted spectacles.

It has become apparent that apparatus, which would allow the assessment of the subject's likely improvement in performance to be made with an enclosure as described in the foregoing paragraph, but prior to the formulation and prescribing of the appropriate tinted glasses, would offer significant benefits.

The current invention enables a tint to be simulated and allows for a simultaneous assessment of the likely improvement in the subject's performance that would result, thereby, prior to prescribing the appropriately tinted lenses, and for a practitioner or the subject himself to carry out such assessment in a manner analogous to that used with the motorised apparatus described in the foregoing.

### SUMMARY OF THE INVENTION

It is an object of the current invention to improve the efficiency of prescribing tinted lenses in order to alleviate symptoms of a variety of visually induced physiological defects and/or pathological conditions.

It is another object of the invention to permit a subject to select a preferred tint for his or her own comfort or enhanced visual performance by having direct control of a simulation of the visual effect of such a tint.

It is another object of the invention to improve a subject's and/or user's comfort and/or performance, when using a range of instruments, the principal function of which is to assess the subject's visual performance.

It is a further object of the invention to provide the means for a user of an optical instrument in which the visual field is artificially illuminated to select the tint of the illumination, so as to optimise user comfort.

Using a specific controllable light source for a particular task can be preferable to other forms of treatment (e.g. tinted spectacles), as the task lighting can be tailored precisely, for example to take account of the ambient conditions. A specific light is also of particular importance in certain eye conditions such as macular degeneration or cataract as optimum performance is directly related to visual stimulus input, particularly if the person has relatively poor vision. Specific stimulus modification will also be of great use in migraine prevention and treatment, with possible uses in attention deficit hyperactivity syndrome and some types of epilepsy. Where it is desirable for the subject to use tinted spectacles, a controllable light source, as described herein, is a useful tool for defining the preferred filter characteristics of the tinted lenses.

Thus, in accordance with the current invention apparatus for the assessment and/or improvement of a subject's visual performance comprises means for presenting the subject with an observable feature against a background; means for changing the location of said feature along at least one locus of points; means for providing an illuminant to illuminate at least said background; and means for controlling the tint and/or brightness of the illuminant, under which, in use, the background together with the feature is observed by the subject, wherein said means for changing location and said means for controlling tint and/or brightness are simultaneously operable.

In preferred embodiments an illuminant is provided by at least two sources, each of which is arranged to emit a respective spectral component of the visible spectrum, wherein a first spectral component has its peak at a wavelength which is located between 510 nm and 540 nm (and preferably between 520 nm and 530 nm) and contributes predominantly to a respective first tristimulus value of the light entering an eye of the subject. Preferably, each spectral component has a spectral power distribution having a width at half height which does not exceed 50 nm.

The tint control means typically comprises means for selecting a weighted mixture of spectral components to provide the illuminant.

The feature may be printed on or supported by a carrier and the means for changing its location may comprise one actuator or two co-operating actuators to provide, in use, motion of the feature along at least one locus of points. Preferably, each actuator comprises a motor. Where two actuators co-operate, the linkage between each motor and the feature carrier comprises one or two belt and/or wire drive members each of which is driven by both motors.

In a preferred embodiment of the invention the illuminant is provided through a light guiding component and the sources are arranged to inject light along an edge of this component. The feature's illuminated background can comprise light emitted from one face of the component. An enclosure with an aperture, through which, in use, the feature is observed by the subject, may be provided, with at least one face comprising a surface for providing the illuminant.

In certain embodiments the feature comprises a dot. In others, it comprises a ball.

Preferably, computing means is provided to control both the tint and brightness of the illuminant and the location of the feature.

In preferred embodiments the subject or a practitioner would have the means to control one or more of the tint of the background, the position of the feature and the motion thereof.

The apparatus of the current invention may be used to assess and/or to improve a subject's visual performance.

### DESCRIPTION OF PREFERRED EMBODIMENTS

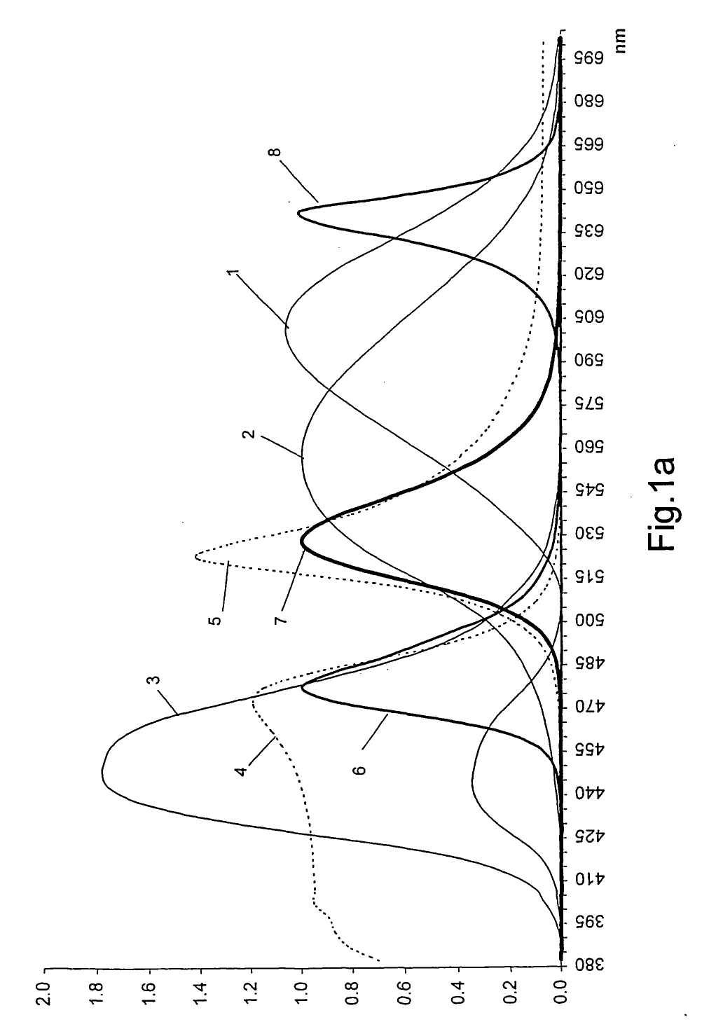

The invention will now be described with reference to FIGS. 1a to 10 in which:

FIG. 1a illustrates the response of the human visual system, as a function of the wavelength of the light incident thereon. Additional curves are provided to aid in the description of the invention.

FIG. 1b provides further curves showing the sensitivity characteristics of the colour receptors or cones at the human retina.

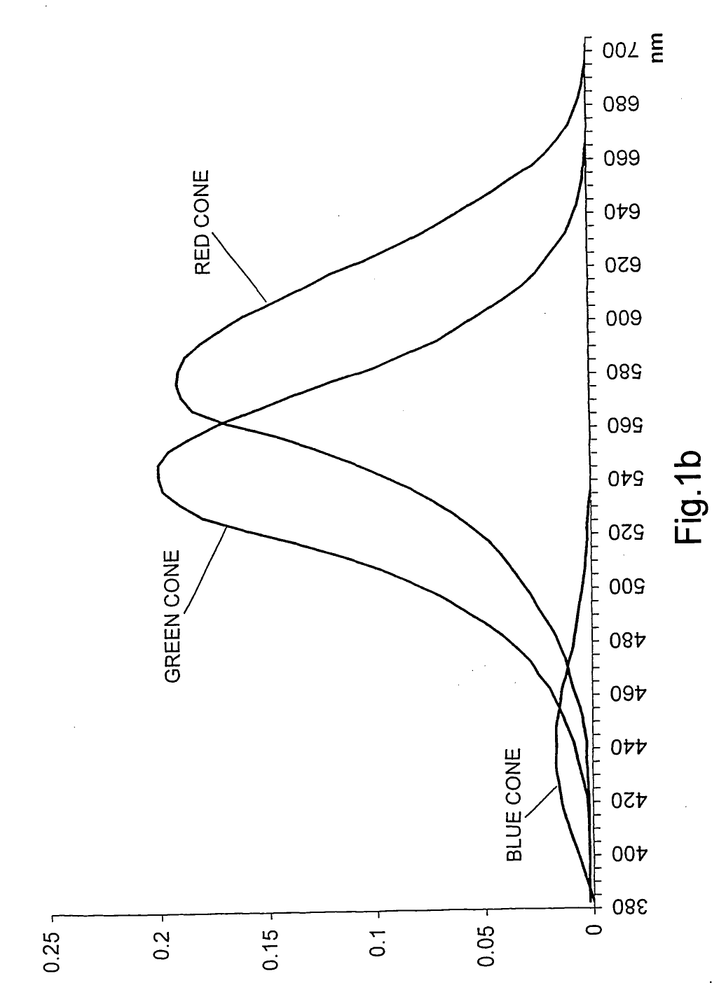

FIG. 2 illustrates, diagrammatically, apparatus constructed in accordance with the invention in order to provide a colour controllable source of illumination,

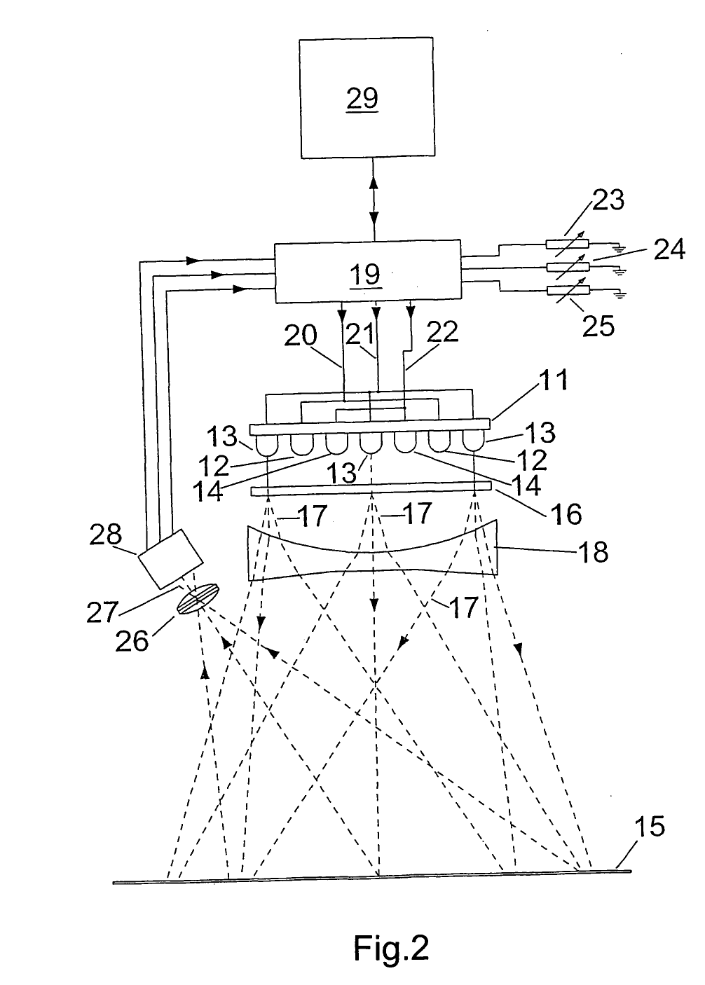

FIG. 3 shows in flowchart form a preferred method in accordance with the invention for use of the apparatus of FIG. 2.

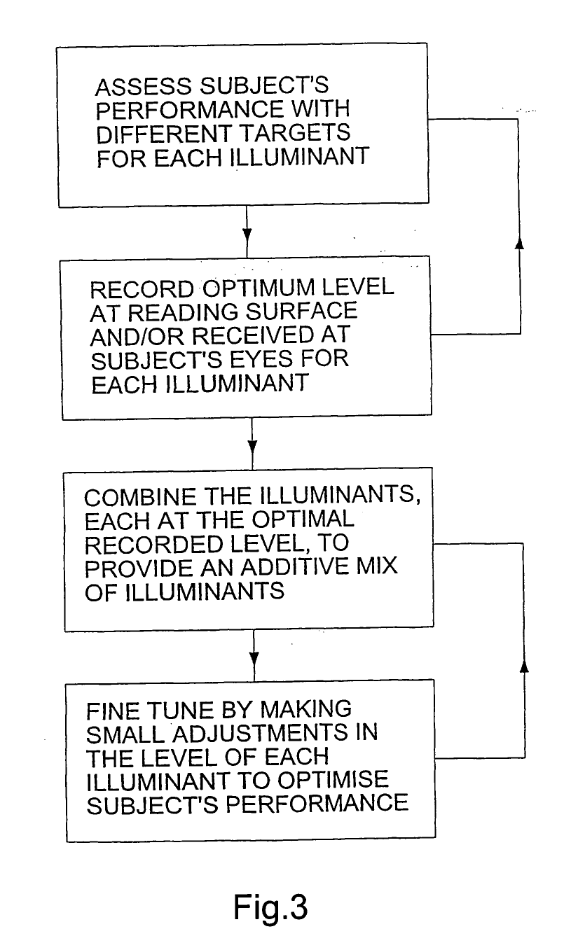

FIG. 4 shows the transmission spectrum of a typically tinted lens, formulated to reduce the relative stimulus to one type of cone, in accordance with the invention.

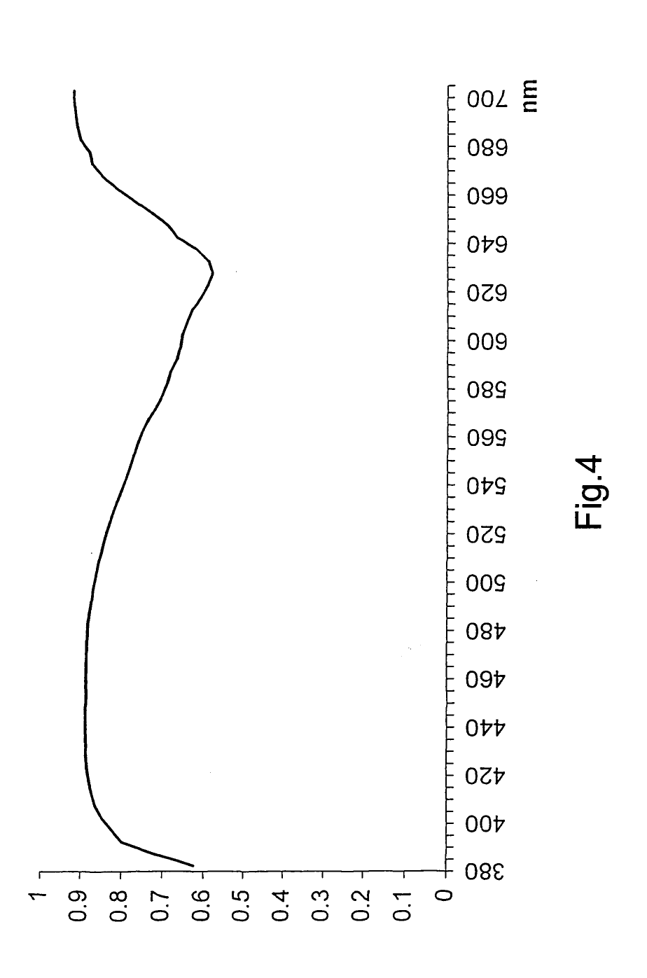

FIG. 5 provides a graphical illustration of colour space and the position of the colour co-ordinates of sources, as used in embodiments of the invention, within this.

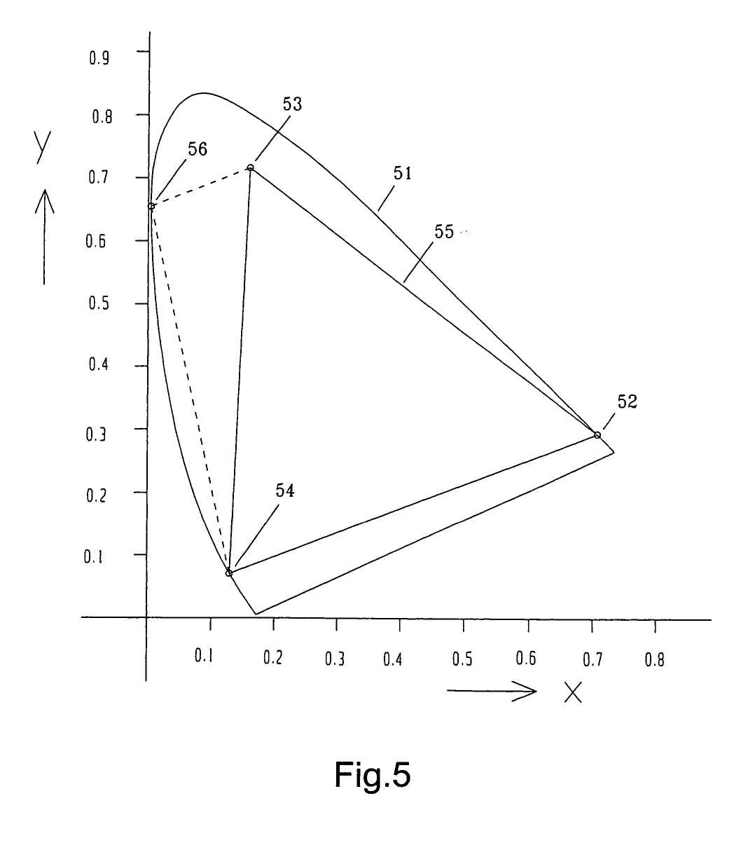

FIG. 6 illustrates an alternative embodiment of apparatus constructed in accordance with the invention.

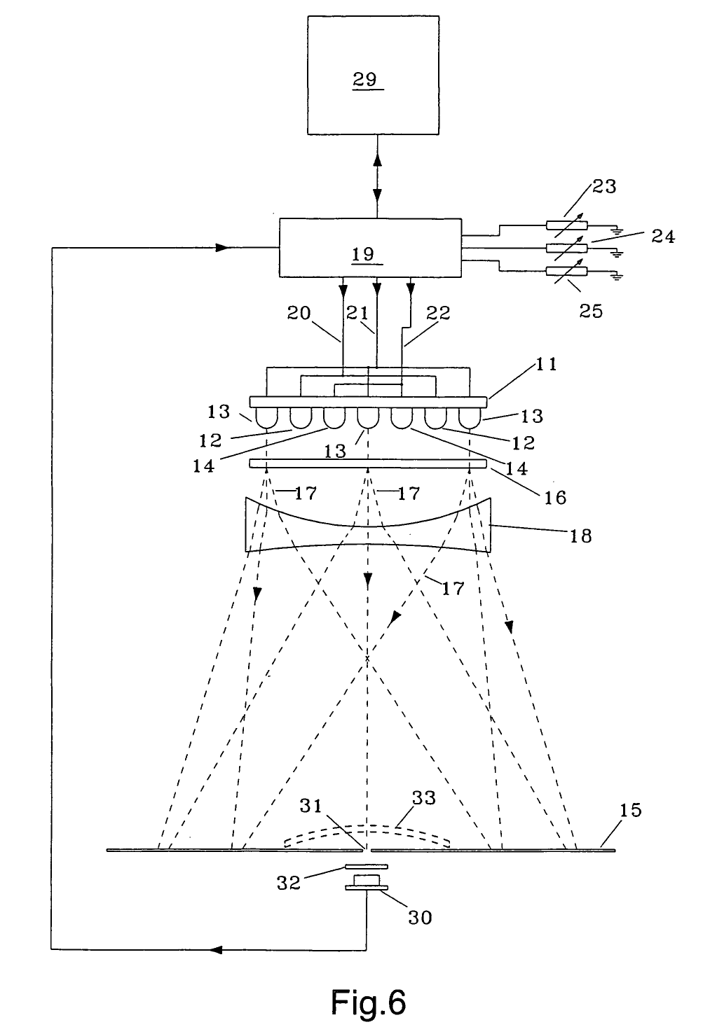

FIG. 7 illustrates a further embodiment of apparatus constructed in accordance with the invention.

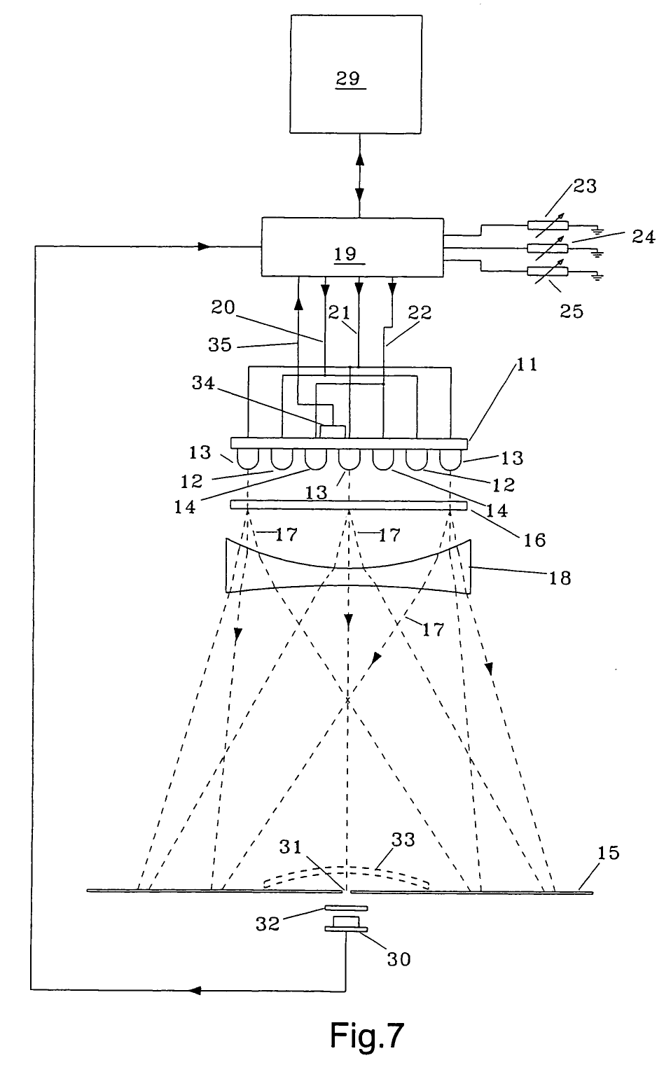

FIG. 8 shows apparatus, inter alia, for assessing convergence and pursuit movement constructed in accordance with the invention,

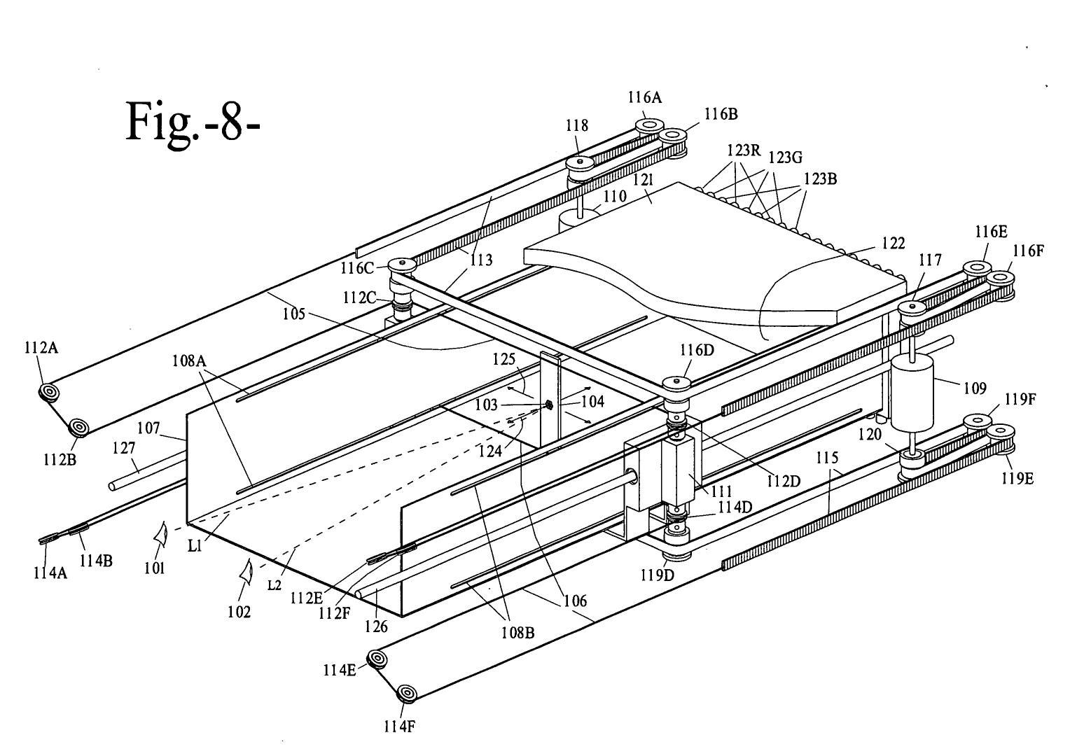

FIG. 9 shows an example of a symbol which may be used for certain visual tests carried out with the apparatus of the invention and

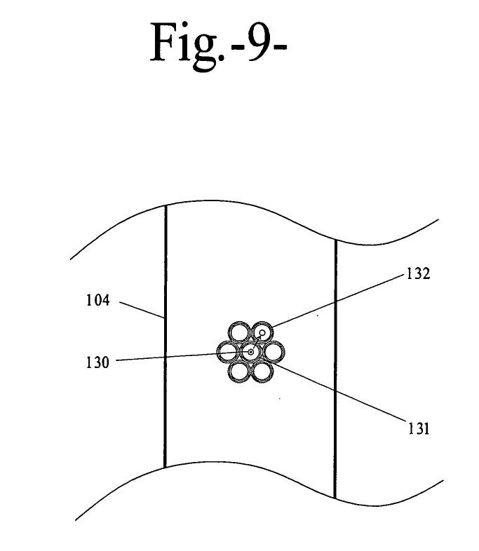

FIG. 10 illustrates apparatus in accordance with the invention for use by an individual, whilst carrying out a self assessment for colour preference.

FIG. 1a shows the so-called spectral tristimulus values as a function of wavelength λ. These curves, which represent the amounts of idealised primaries required to match any of the pure spectral colours in the visible range and are related to the colour sensitivity characteristics of the human eye. Curve 1, typically designated as the function \overline{x}(λ), primarily comprises the responsivity of the red sensitive cones of the human retina. The blue sensitive cones' responsivity is, suitably scaled, also included in this first tristimulus curve (see FIG. 1b). Curve 2 is, to a good approximation, a summation of the green and red cones' responsivity curves and is designated as the function \overline{y}(λ) and actually corresponds to the overall spectral sensitivity of the eye. Curve 3 essentially comprises the blue cones' spectral sensitivity characteristic \overline{z}(λ). It will be clear from these curves that the \overline{x}(λ) curve has a subsidiary maximum in the blue region of the visible spectrum. A colour stimulus to the human visual system may be conveniently expressed as three values, the so-called tristimulus values (X, Y and Z), each of which involves an integral over the visible spectrum of the spectral power distribution reaching the retinal cones convolved with the respective tristimulus curve. For example:

$$X = {\int }_{λ}^{} P ( λ ) \overline{x} ( λ ) ⅆ λ$$

Two further sets of curves are shown in FIG. 1a. One of these comprises dashed lines 4 and 5. Line 4 represents, following some normalisation, the ratio between \overline{z}(λ) and the root mean squares of \overline{x}(λ) and \overline{y}(λ) and line 5 represents, on the same basis, the ratio between \overline{y}(λ) and the root mean squares of \overline{x}(λ) and \overline{z}(λ).

The objective in calculating these merit functions is to find those points within the visible spectrum where the effect of the resultant stimulus of the human visual system is substantially expressed as a change to one of the tristimulus values, with the change to the other two being minimised relative thereto. What the two curves show is that, for a maximum change to Z relative to X and Y, stimulation of the human visual system at a wavelength of around 470 nm should be used and that, for maximum change of Y relative to X and Z, stimulation of the human visual system at a wavelength of around 520 nm is most effective. The purpose of the merit function is to find the optimal wavelength for maximising Y, relative to X and Z. Its value peaks near 520 nm, and drops to half its maximum at approximately 510 nm and also at 540 nm. A choice of wavelength within this range would be acceptable, though, for best results, a wavelength between 520 nm and 530 nm should be chosen. There is no clear choice for X, but a wavelength of around 640 nm is found to achieve good red saturation without too much loss of overall sensitivity.

It is an objective of this invention to provide a means for controlling the colour stimulation of the human visual system, so that an optimum ratio of X, Y and Z values can be established. When this is achieved, the visual or related disability and/or symptom of the subject, experienced under normal illumination, can be substantially alleviated. It will be clear that a combination of controllable narrow-band light sources, located respectively at substantially 470 nm, 520 nm and, say, 640 nm, will readily achieve this goal. All of these wavelengths are substantially achieved with commercially available LED's, the bandwidths of which typically vary from 17 nm to 47 nm. Typical examples of such emitted spectra are shown in FIG. 1a as curve 6, for Z, peaking at 470 nm (defined as blue herein), curve 7, for Y, peaking at 524 nm (defined as green herein) and curve 8, for X, peaking at around 640 nm in the red portion of the spectrum. The red wavelength is not as critical as the other two, for the reasons stated above.

By combining the light from the three different types of LED, as specified above, a wide range of colours can be achieved. A lamp comprising one or more of each type of LED, arranged in a variety of different ways, in which each group of a specific colour is controlled by an adjustable signal, can be used to optimise the illumination for a given subject carrying out a specific task, such as reading or writing. For example, a person who suffers from dyslexia may have a reading difficulty significantly alleviated by the partial or complete exclusion of the red illumination, in effect, by reducing the stimulation of the red sensitive cones.

Embodiments of the current invention use a multi-colour light emitting diode (LED) array, operated within an optical assembly so that colours can be mixed to create the optimum lighting for any patient. An array of different coloured LEDs, typically red, green and blue, in accordance with the principles outlined above are operated either individually or together, so that it is possible to select single primary colours or combine the various LEDs to give different hues and illuminance. The primary advantage with this type of lighting being that it can be used for both reading and writing.

In practice, each LED type (red green or blue) has its own chromaticity co-ordinates and the differences between that of one type and of the other two determine the range of colours that can be achieved by appropriately combining their outputs.

The table below sets out typical values of x, y and z (in which z is defined as 1-x-y) for each of the three LED types

| | x | y | z |
|---|---|---|---|
| Red | 0.706 | 0.294 | 0.000 |
| Green | 0.159 | 0.717 | 0.124 |
| Blue | 0.129 | 0.071 | 0.800 |

A method, well established in the prior art, for depicting a particular colour within a continuum of possibilities is to represent this as a point on a chromaticity diagram of x against y. In such a diagram (see FIG. 5, which depicts the CIE 1931 chromaticity diagram), all possible colours fall within a defined and closed locus of points 51. In practice, each LED will have an xy co-ordinate, falling on or within this boundary, and the colour co-ordinates 52, 53 and 54 of each of the three types of LED, between them, define a triangle 55 within this complete colour space. The larger this triangle, the greater the range of colours which can be produced by varying the contributions to the illuminant from each of the LED types. For each target value of x and y, there will be a defined output requirement from each type of LED. It may be shown that, after inverting the matrix comprised of the three colour co-ordinates (each having three terms) of the red, green and blue LED's provided above and after applying suitable compensation factors to each drive of the LED types, amongst other things, to compensate for their spectral distributions and quantum efficiencies, a 3×3 matrix may be constructed, which defines the required demand to apply to each of the primary sources in order to obtain a specific point (x, y, z) in colour space. This matrix is of the general form and is used as follows:

$$[ \begin{matrix}Red\_demand \\ Green\_demand \\ Blue\_demand\end{matrix} ] = [ \begin{matrix}1.095 & - 0.215 & - 0.158 \\ - 0.634 & 1.543 & - 0.033 \\ 0.063 & - 0.151 & 0.796\end{matrix} ] \times [ \begin{matrix}x \\ y \\ z\end{matrix} ]$$

What the above three relationships define is that, in this particular example and for a target white illuminant (x=0.333, y=0.333, z=0.333) to be provided, relative demands of 0.241 from the red source, 0.289 from the green source and 0.236 from the blue source are required. If the chromaticity co-ordinates of the red source (0.706, 0.294, 0) are applied to the right hand side of the above equality then, as expected, the only demand required is that of the red source. The three LED types 12, 13 and 14 have chromaticity co-ordinates, depicted in FIG. 5 as points 52, 53 and 54. As stated above, theses define a triangle 55 within the closed locus of points 51 in the chromaticity diagram which represents the continuum of all colours. If chromaticity co-ordinates which fall outside this triangle are applied to the right hand side of the above equality, a negative demand from at least one of the primary sources would be indicated. This is not possible and consequently the triangle defines those colours which the colour selectable lamp of this invention can typically provide. It is possible, within the scope of this invention, to introduce additional narrowband light sources, such as, for example, a narrow band source having its peak at 505 nM and having colour co-ordinates (x, y, z) of (0.004, 0.655,0.341). This is shown as point 56 in FIG. 5. By defining a second matrix, we may calculate the demands required from the original green and blue sources together with that from this new blue-green source, in order to access that portion of colour space bounded by the triangle defined by points 53, 54 and 56.

In practice the narrow-band sources used in preferred embodiments of this invention and their particular position in colour space provide a very large gamut of possible colours. A colour selectable lamp constructed in accordance with this invention allows much greater flexibility than that of systems which employ subtractive broadband filters to control the colour of the illuminant and provides the opportunity to better taylor the illuminant to each user. This could have important applications in the office and school environment where ambient lighting limitations contribute to reading and writing problems for some individuals.

Turning to FIG. 2, this shows diagrammatically how a number of components may be combined in accordance with the principles of the invention to form a colour controllable light source.

A lamp 11 comprises an array of LED's. The array includes red emitters 12, having an emission spectrum peaking at 640 nm, green emitters 13, having an emission spectrum peaking at 524 nm, and blue emitters 14, having an emission spectrum peaking at 470 nm. The LED's are distributed in such a manner that the field illuminated by each type at a reading surface 15 is approximately the same. In order to ensure that there are no substantial differences in the mix of colours at any given point on the reading surface, a diffuser 16 is placed in the path of the emitted light. This diffuser may take several different forms. A lenticular screen or microlens array is found to be effective, as well as other kinds of efficient light scattering media. For example, a material comprising changes of refractive index over short distances can be very effective.

The effect of distributing the individual LED's in an even manner, together with the action of the diffuser 16, is to provide a very even mix of light at the reading surface 15. In order to extend the effective area of illumination, a divergent lens assembly 18 can be very useful. Although this is shown as a conventional meniscus lens, a compact equivalent, such as a fresnel lens may also be used.

A control unit, typically a microprocessor, 19 receives a number of different inputs, prior to driving each group of LED's via outputs 20 for blue, 21 for green and 22 for red. At its simplest level, variable resistors 23, 24 and 25 are used to set the light output from the red, green and blue LED's respectively. The components identified, thus far, comprise a colour controllable lamp. This can be used by a subject to select a particular combination of red, green and blue illuminants, which is optimal for his or her reading or writing performance.

In practice, a more sophisticated version of such a lamp would adapt the light output demanded from the LED array to take account of the ambient conditions. In FIG. 2 a lens 26 forms an image on the receiving surface 27 of a camera 28. This may be a CCD or other photo-detector array, behind a colour filter array. Using known principles, the video signal from the CCD can be analysed to provide a reading of the level of illumination at surface 15, in addition to its colour mix. There will be a specific matrix, which will allow the measure of light passing through each component of the camera's colour filter array to be translated into a red, green and blue LED light combination. Some of this will be contributed by the ambient light impinging on surface 15. The output, required from each type of LED, is adjusted by control unit 19, accordingly. As a consequence of the use of camera 28 to monitor the illumination of surface 15 the resulting system will also be stabilised against other variations, such as changes in the efficiency of the optics or LED's.

The apparatus of FIG. 2 can be very useful as a diagnostic tool, particularly when used in conjunction with a computer, shown as block 29. Amongst other things, the computer can be used to store the selected tint of the illumination at surface 15, when this has been optimised for the subject.

Turning to FIG. 3, this outlines, in summary form, a methodology in accordance with the invention for establishing the optimal illumination for a specific subject, such as, for example, a person suffering from visual dyslexia.

The first step in the procedure is to determine the best illumination conditions for a variety of different reading tests. This is done by illuminating the reading material at surface 15 of FIG. 2 with one of the illuminants. This is increased in brightness, until the subject is satisfied that the optimal brightness has been found. It may be necessary to pass through the optimum and to reduce the brightness slightly to establish that setting. This step is repeated for each of the illuminants (LED groups), separately. It is quite possible that the optimum level for the red illumination may be at 50% of maximum, for a particular subject, whereas the green and blue illuminants would be quite acceptable at their maximum levels. The particular settings for each illuminant will be highly subject dependent. Step 2 is to record the optimum level for each illuminant, either directly from the controls or transferred automatically to a computer.

Once the individual optima have been established, the recorded levels of each primary illuminant are combined in Step 3 of the procedure. Step 4 is to fine tune this mixture by making small adjustments to each primary (red, green and blue), in small steps, until an optimum mix is established for the subject. The step changes would be made in both directions, decreasing or increasing the particular illuminant, and establishing whether there is an improvement or otherwise in the subject's performance. By iteration of Steps 3 and 4, the best combination is found.

One of the key objectives of this invention is to use the arrangement of FIG. 2 as a diagnostic tool, in order to arrive at an optimal formulation for the filters to be provided for the lenses of spectacles or contact lenses to be worn by the subject. The colour of the light reaching the subject's eyes is recorded by the system of FIG. 2 and stored in computer 29. This record will typically contain information about the settings of the LED sources and, if any, the colour and level of the ambient illumination at the time that the measurements were made. By prior knowledge or use of colour camera 28, any colouration of the reading surface 15 may also be accommodated.

In practice there will be a finite selection of filter formulations available. A typical filter characteristic is shown in FIG. 4. Curve 41 represents the percentage transmission of a red absorbing (blue tinted) filter as a function T(λ) of the wavelength λ of the light incident upon it. Our interest is in knowing what the response at the retina of each eye will be for each of the cones when the subject views material through this filter. In order to calculate this we must multiply each of the tristimulus curves at every wavelength with the spectral distribution of the light arriving at the retina and integrate this result over the visible spectrum. The result will be one of the tristimulus values for the particular tint, as defined by the CIE 1931 chromaticity diagram (as shown in FIG. 5). It will comprise a number of components, including the following:
1) the spectrum of the illumination which the subject will use when reading or writing (This could be daylight or light from a tungsten or fluorescent lamp and each will have a different spectrum),
2) the background reflectance spectrum of the material being read and
3) the relevant tristimulus curve.

For the response corresponding to each of the tristimulus values the integral required will be of the form

$$X = {\int }_{380 nm}^{780 nm} I ( λ ) T ( λ ) R ( λ ) \overline{x} ( λ ) ⅆ λ ,$$

Where I(λ) is the illumination spectrum, T(λ) is the filter's transmission spectrum, R(λ) is the illuminated substrate's reflectance spectrum and \overline{x}(λ) is the relevant tristimulus curve, shown, suitably normalised as curve 1 in FIG. 1a. Two further integrals would be calculated for the Y and Z tristimulus values.

It will be clear to those versed in the art that the same tristimulus values can be achieved with a different illumination spectrum and, in principle, without the use of the intervening transmission filter. Indeed, where the illumination spectrum is comprised of the combination of the three primary illuminants provided by the red, green and blue LED's of FIG. 2, this spectrum will have three well-defined peaks. As already explained, by reference to FIG. 1a and FIG. 1b, each of these peaks will have a particularly significant influence on only one of the tristimulus values.

It is a further objective of this invention to simulate the effect of any particular filter by providing illumination which simulates the effect on the visual system that would result from the use of that filter under the expected lighting conditions. Thus the LED outputs, with the reflectance characteristics of the reading surface 15 in FIG. 2 being taken into account, must be adjusted to simulate that part of the function under the integral above represented by I(λ)T(λ)R(λ). In effect, I(λ)T(λ) will be replaced by the following expression:
E(λ)=rR(λ)+gG(λ)+bB(λ),
where r, g and b represent the components of each of the primary illuminants and R(λ), G(λ) and B(λ) are the respective spectral power distributions of these, as shown in FIG. 1a as curves 8, 7 and 6 respectively.

For every choice of filter characteristic available there will be values of r, g and b which will simulate the effect for the subject under a particular selection of lighting. Having established an optimal tristimulus value for the subject by using the procedure of FIG. 3, a best choice of tint may be selected or formulated. A database of all standard filters may be held on computer 29, in order to provide a convenient method for prescribing an available choice of filter. The precise effect of that filter being available for the subject to experience by simulation using the apparatus of FIG. 2

It follows from this that the apparatus of FIG. 2 may be used to determine the relative colour response of an individual's eye. In this case a surface of known colour reflectance is made to look white by adjusting r, g and b values above. The expression describing this is:
CC[surface(λ)*(Eᵣ(λ)*rR(λ)+E_{g}(λ)*gG(λ)+E_{b}(λ)*bB(λ))]=CCₚ
where CC[f(λ)] is the colour co-ordinate transformation of a spectrum, CCₚ is the perceived white colour response and Eᵣ(λ), E_{g}(λ) and E_{b}(λ) are the eye responses. For a known surface and instrument settings and a normal eye response then the perceived white colour will correspond with the actual colour co-ordinates of white with CCₚ=[0.33,0.33,0.33].

For an eye with a different colour response CCₚ will be at a different position in colour space and the vector between this position and nominal white will be a measurement of relative colour response of the eye.

By further reference to FIG. 1a it also follows that, in order to reduce the X tristimulus value to a minimum, a light source with its energy concentrated at around 505 nm is required. Such a facility may prove particularly useful in circumstances where the function of the lamp is a diagnostic one and a complete absence of the X stimulus is desired. Its provision, as illustrated earlier herein, will also increase the range of tints which can be simulated by apparatus constructed in accordance with the invention.

Although the embodiment of FIG. 2 incorporates a divergent lens to spread the illumination over the desired area, this is not an essential component for the operation of the lamp, as the combination of a diffuser and suitably positioned LED's can be chosen to illuminate any specific area. Whilst the embodiments illustrated herein utilise LED's with relatively narrow-band emission spectra, other devices such as laser sources may be used as alternative illuminants. Furthermore, whereas a camera 28 is employed to analyse the colour of the illumination of surface 15, this could, in practice, be replaced by a series of photodiodes receiving light from this surface through suitable colour filters.

An alternative embodiment of the invention is illustrated in FIG. 5. This is similar to the embodiment of FIG. 2, but, instead of a CCD camera to view the light scattered from reading surface 15, a photocell 30, having precisely known spectral sensitivity, is positioned behind a small aperture 31 in surface 15 at which material to be viewed under a colour controlled illuminant would, in normal use, be placed. A diffuser 32 is placed immediately in front of photocell 30 to ensure that it responds uniformly to light from lamp 11, regardless of its point of origin at the lamp. Another optical arrangement to achieve this end result would comprise a lens (not shown) positioned between aperture 31 and photocell 30 and arranged to image the lamp onto the photosensitive area of photocell 30. The function of photocell 30 is two-fold. It is used within an automated calibration procedure to adjust the respective drive currents to the red 12, green 13 and blue 14 LED's, in order to provide the correct balance for a white illuminant. Each LED type is activated in sequence and the power adjusted to ensure that the expected response, which can be calculated from the known spectral output of the LED and the corresponding spectral sensitivity of photocell 30, is received by the latter. Once the LED's have been balanced in this way, they may be used in conjunction with photocell 30 to test the transmission characteristic at three points of the spectrum of any lens (shown in broken line format as item 33 in FIG. 5) which has been formulated using a known filter material. For a given filter material the ratios of the three responses will be known and the density of the filter will be calculable. The combination of the selectable LED's and known photocell characteristic, enables a precise validation of transmission characteristics of lens 33 to be carried out.

An embodiment of the invention which includes temperature compensation to improve precision is illustrated in FIG. 7. A temperature sensor 34 is included and is attached to the assembly of lamp 11, which incorporates the LED's. The temperature of this assembly is relayed via line 35 to microprocessor 19. It has been established that the quantum efficiency of an LED typically changes as a function of its operating temperature and some loss of light output may be expected as the device's temperature increases. This effect can be effectively offset by adjusting the demand to the LED as a function of temperature and line 35 provides microprocessor 19 with the necessary means for doing so.

An embodiment of the invention which permits a subject's visual stability to be tested in respect of moving objects, or for different degrees of convergence of the eyes, is now illustrated with reference to FIG. 8. The apparatus of FIG. 8 has been drawn diagrammatically, without its chassis or cladding, so that its key components are clearly visible and its function can be more readily understood.

The subject, represented by eyes 101 and 102 and whose visual stability is to be assessed, is asked to observe a feature in the form of symbol 103, which may be as simple as a dot, printed on a transparent carrier 104. Carrier 104 is suspended by two wires 105 and 106, preferably made of a transparent material such as nylon, within an enclosure. The enclosure is open at one end and one of its components is a tray 107 having two sides and a base each of which is painted white. In order to gain access to the enclosure, each wire passes through two opposing horizontal slots 108A and 108B in respective sides of tray 107.

Wires 105 and 106 are driven together, through associated belts 113 and 115 and by the action of two motors 110 and 109, so as to move carrier 104 under control of a computer (not shown). Each wire passes over a series of six pulleys, which are free to rotate on their respective axes and two of which are mounted on a movable carriage 111. Wire 105 passes over pulleys 112A, 112B, 112C, 112D, 112E and 112F and is connected to a toothed belt 113 at both ends in order to form a continuous drive loop. Pulleys 112C and 112D are mounted respectively on carriage 111. Likewise, wire 106 passes over pulleys, five of which are shown as 114A, 114B, 114D, 114E and 114F, and is connected to toothed belt 115 at both ends, in order to form a second drive loop. Again, pulley 114D and its opposite counterpart are mounted on carriage 111. Each drive loop operates in two planes, one of which allows its respective wire to enter the enclosure through its associated slots and, a second plane, which allows its respective belt to pass above or, as the case may be, below the enclosure. Transition from one plane to the other is accomplished by a 30° tilt of the axis of each wire guiding pulley, which is not mounted on carriage 111, relative to the direction of the axes of the four pulleys, that are.

As is the case for each wire, each belt passes over a series of six pulleys which are free to rotate on their respective axes and two of which are mounted on movable carriage 111. In addition, each belt passes over two toothed pulleys each of which is driven by a respective motor. Belt 113 passes over freely rotating pulleys 116A, 116B, 116C, 116D, 116E and 116F. It is driven by pulley 117 attached to the shaft of motor 109 and pulley 118 attached to the shaft of motor 110. Pulleys 116C and 116D are mounted on carriage 111 and are free to rotate about the same axis as pulleys 112C and 112D respectively. Belt 115 is arranged in similar fashion to belt 113, with six freely rotating pulleys, three of which 119D, 119E and 119F are shown, and two drive pulleys, one of which 120 is shown attached to the shaft of motor 109.

In the embodiment of this invention illustrated by FIG. 8, the top and rear surface of the enclosure comprise edge lit plastic slabs 121 and 122 respectively. The function of these slabs, which operate in a similar manner to the back lights used with many types of visual display, such as flat panel LCD monitors, is to distribute the light injected along the edge of the slab to exit uniformly from its larger surface area and, thereby, to illuminate the enclosure and its contents in an isotropic manner. Top slab 121 has been cut away to allow illustration of the components within the enclosure. Belt 113 passes above the plane of slab 121, whilst wire 105 passes below this.

The difference between the edge lighting of conventional back lights, which typically comprise a long thin fluorescent tube, and the current illumination arrangement is that the fluorescent tube is replaced in the embodiment of FIG. 8 by an array of three different types of light emitting diodes (LED's) comprised of red emitters 123R, having an emission spectrum peaking at 640 nm, green emitters 123G, having an emission spectrum peaking at 524 nm, and blue emitters 123B, having an emission spectrum peaking at 470 nm. Suitable distribution of scattering features on both sides of plastic slabs 121 and 122, together with a reflective sheet placed at each back surface and a diffusing sheet at each front surface, as is well known in the prior art, ensures a uniform distribution and mix of the light. Depending on type, the width at half height of the spectrum emitted by each LED varies from 17 nm to 47 nm and would typically not exceed 50 nm.

Control of the output of the LED's in accordance with the foregoing provides an evenly distributed illuminant within the enclosure and a wide choice of well defined tristimulus values for the subject. In addition to providing illumination with well defined colour co-ordinates, the LED control mechanism may be programmed to simulate the time varying characteristics (such as flicker) of light sources such as fluorescent tubes and the like.

Operation of the apparatus now proceeds as follows. The subject positions his eyes 101 and 102 at the front of the enclosure and attempts to converge the line of site of each eye L1 and L2 in order to view the target (symbol 103) on carrier 104. Under control of the subject or the practitioner and with the help of a small control unit (not shown), the position of the target can be changed in two directions both forward and backward or longitudinally 124, along the Z ordinate, and laterally 125, along the X ordinate. The way that the mechanism of FIG. 8 is used to achieve this is that motors 109 and 110 co-operate to move carrier 104 along each of the X and Z ordinates. The X ordinate is altered by operating the motors in the same direction, whilst the Z ordinate is altered by a contra-rotation of the motors. For motion along the X ordinate, carriage 111 remains stationary, because the length of the section of toothed belt 113 between pulleys 117 and 118 remains constant. However, when motors 109 and 110 have opposite directions of motion, this section of belt 113 (as well as that of belt 115) increases or decreases in length, compensated by a corresponding respective decrease or increase in the length of the section of its associated wire 105, between pulleys 112B and 112E. Accordingly, the carriage slides along two shafts 126 and 127, which pass through matching bushes on carriage 111.

The current invention allows the carrying out of procedures which have not been practical before. The practitioner can vary both the brightness and the chromaticity co-ordinates of the illuminant within the enclosure, to find the subject's optimum position in colour space or as a second step to simulate the tint, were the subject to view the target through a proposed prescription tinted lens under a given (standard) illuminant. Under these conditions the subject's ability to perform a variety of visual tasks can be tested, such as his ability to converge centrally or to the right and left of the midline. Such tests can be carried out at a variety of distances and, whilst being undertaken, the simulated tint can be varied in order to find that prescription which, for example, would optimise the subject's visual stability.

Although not described in detail, within the preferred embodiment of FIG. 8, a variety of tests may be performed with apparatus as described herein. For example, instead of a simple dot as the target to be viewed, a small pattern or symbol such as that shown in more detail in FIG. 9 may be provided as the target on carrier 104. As the distance of this pattern from the subject is varied, it is quite common that anomalous visual effects are stimulated, both as a function of changes in colour and distance. A typical effect would be that the central dot 130 is perceived to move from the center of the central circle 131 to that of one of the surrounding ones 132, or that it disappears altogether.

In some tests, including those involving a simple dot as the target, the latter is designed to move, and the speed and direction of movement of the target in a plane perpendicular to the line of sight of the observer (along the X ordinate) is varied. Other tests involve patterns or strings of readable text, which have substantial lateral extent. A wide carrier for such graphics would be used and the lateral motion option of the apparatus would be inhibited. The tests would, inter alia, include the facility to test the subject's visual field, as a function of the illuminant's colour co-ordinates. In such tests, the perpendicular distance of the plane of the image from the observer is typically adjusted so that the observer can make judgements as to the clarity or integrity of the image at different distances from the eye.

Whilst the current invention, in the preferred embodiment of FIG. 8, comprises edge lit illumination panels in the form of slabs 121 and 122, it will be clear to those versed in the art that other arrangements are practical and would fall within the broad scope of that which is claimed herein. For example tray 107 could be provided with a back surface which is also painted white. This tray could be lit from any source of light, the chromaticity co-ordinates of which may be varied in line with the principles of this invention and which provides an adequately even distribution of light for viewing of the target by the observer.

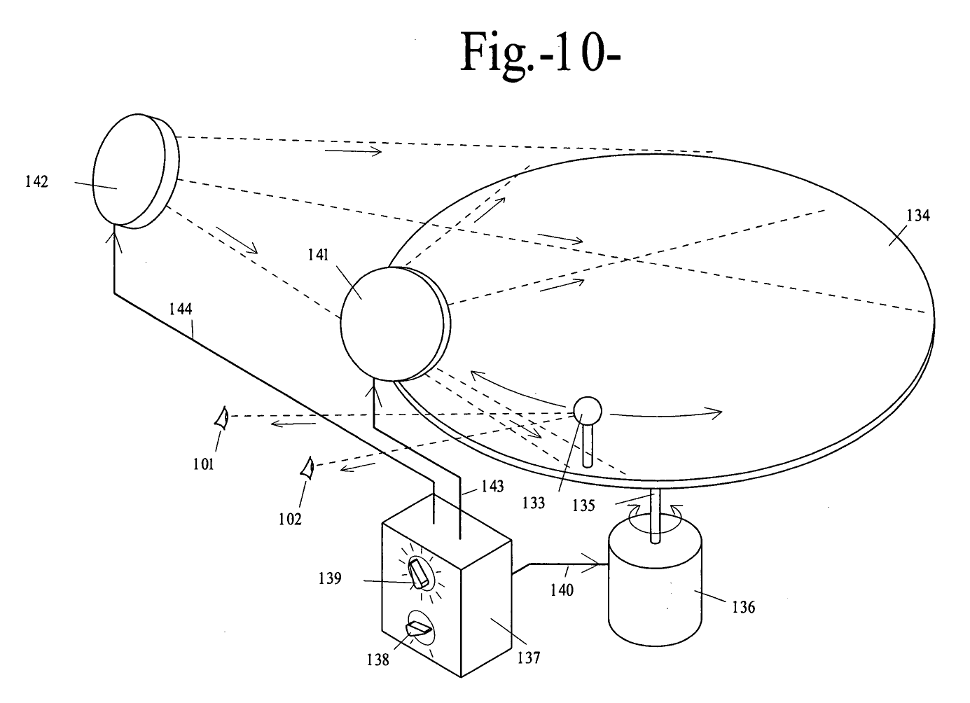

A second embodiment of the invention is now described with reference to FIG. 103. As with FIG. 8, the apparatus of FIG. 10 has been drawn diagrammatically, without its chassis or enclosure, so that its key components are clearly visible and its function can be more readily understood. A viewing window, set in the front face of the enclosure, through which the space within it may be viewed, has also been omitted from the drawing. The apparatus is intended for use by an individual who wishes to select his (or her) preferred tint, from a predefined set of tints. An optimal selection could enable a subject to improve his co-ordination in ball sports or, quite simply, to improve his ability to visually track a moving ball, as a spectator. The user positions himself to view a feature in the form of a ball 133 with his eyes 101 and 102. Ball 133 is mounted on a rotating stage 134. The motion of stage 134, which is mounted on a shaft 135, is effected by a motor 136. The speed of motor 136 may be pre-set or is controlled by the user, via a control unit 137. Control unit 137, typically comprising a small micro-processor as computing means, has two dials 138 and 139. Dial 138 allows the speed of rotation of stage 134 and its direction to be set, via a cable 140 (The cable comprises several electrical conductors, for example, four would typically be required for a stepper motor). Dial 139 has a number of positions, each of which selects a specific tint of illumination. The illumination is provided by two colour controllable light sources 141 and 142. Sources 141 and 142 are respectively controlled via cables 143 and 144, from control unit 137. For each of the required spectral components of the illuminant, each of the sources 141 and 142 typically comprises one or more LED's as light emitters. Cables 143 and 144 each carry at least two and typically three separate control signals to their respective sources. Each tint selected will require a specific combination of red, green and blue levels of light, which comprise the spectral components. Dial 139 is arranged to select the specific combination required.

The way in which the user proceeds to make his tint selection is as follows. Having selected a tint with dial 139, he fixes his gaze on ball 133. The ball moves along a circular path or locus of points, as stage 134 rotates. The colour of ball 133 may be white, for example, simulating that of a football and the colour of stage 134 green, simulating that of grass. Alternatively, ball 133 might be coloured yellow, as would be appropriate, if the environment to be simulated is that of a tennis court. Where other environments, such as a white ski slope or a red clay tennis court, are to be simulated, other colours for stage 134, ball 133 and, indeed, the inside of the enclosure of the apparatus may be provided. As the user maintains his gaze on ball 133, he can select different tints of illumination, by using dial 139 and may, at his option simultaneously or sequentially, vary the speed and direction of motion of the ball. In this manner, the user can readily establish, with which tint the process of visual tracking or pursuit of ball 133 is most comfortable and his performance optimised.

Typically, the apparatus of FIG. 10 would be used in a point of sale environment, where a selection of tinted spectacles are available for enhancing visual performance or comfort. The purchaser would make his selection by simulating the effect of his prospective choice, using the apparatus described herein. There will be a corresponding tint of illumination for each available set of tinted spectacles under predefined ambient lighting conditions, such as “a sunny day”, “a cloudy day”, fluorescent lighting or tungsten lighting.

From the above descriptions of specific embodiments of this invention, it will be clear to those versed in the art that the principle of incorporating a colour controllable light source in an instrument so that the visual performance and/or comfort of the user or subject may be optimised, is not limited to the particular embodiments described. Any other instrument or related procedure, the principal purpose of which is to assess a subject's response or performance, including that of the refractive characteristics of the eye, and which comprises illumination of a visual field by use of an artificial light source, may, in principle, be improved by application of the principles of this invention. Procedures and related instruments, which would qualify, could include visual acuity analysis procedures, central and peripheral visual field analysers, fusional reserves analysis procedures, eye motion analysis equipment, squint correction procedures, vestibular response analysis, refractive prescribing equipment and, in some cases, auditory testing procedures.

It will also be clear that, whilst the embodiments of colour controllable light sources described employ LED's as their light emitting elements, other sources of light, in combination with suitable transmission filters, may also be suitable under conditions where such arrangements can generate the range of illumination tints required.

Whilst the embodiments of FIGS. 8 and 10 employ physically moving mechanisms which support an observable feature, such movement may be simulated by using a display medium, such as a liquid crystal display, on which such a feature is generated with its own motion under control of a suitable software program.

It will be clear to those skilled in the art that the manufacture of any tinted lens, which is formulated as a result of a prescription derived from the simulation of such lens using apparatus and method constructed in accordance with the teachings of this invention, is the intended end product of such simulation and thereby falls within the scope of the invention.

The invention having been disclosed in connection with the foregoing variations and examples, additional variations will now be apparent to persons skilled in the art. The invention is not intended to be limited to the variations specifically mentioned, and accordingly reference should be made to the appended claims rather than the foregoing discussion of preferred examples, to assess the scope of the invention in which exclusive rights are claimed

1. Apparatus for the assessment and/or improvement of a subject's visual performance comprising means for presenting the subject with an observable feature against a background; means for changing the location of said feature along at least one locus of points; means for providing an illuminant to illuminate at least said background; and means for controlling the tint and/or brightness of the illuminant under which, in use, the background together with the feature is observed by the subject, wherein said means for changing location and said means for controlling tint and/or brightness are simultaneously and/or sequentially operable.

2. Apparatus as claimed in claim 1 in which the illuminant providing means comprises at least two sources, each of which is arranged to emit a respective spectral component of the visible spectrum, wherein a first spectral component has its peak at a wavelength which is located between 510 nm and 540 nm and contributes predominantly to a respective first tristimulus value of the light entering an eye of the subject.

3. Apparatus as claimed in claim 2 in which each spectral component has a spectral power distribution having a width at half height which does not exceed 50 nm.

4. Apparatus as claimed in claim 2 in which the tint control means comprises means for selecting a weighted mixture of spectral components to provide the illuminant.

5. Apparatus as claimed in claim 1 in which the feature comprises a pattern or symbol printed on or supported by or an object supported by a carrier and the means for changing location comprises at least one actuator.

6. Apparatus as claimed in claim 5 in which the means for changing location comprises at least two actuators and one axis of motion of the feature is effected by co-operation between said at least two actuators.

7. Apparatus as claimed in claim 6 in which each actuator comprises a motor and the linkage between each motor and the carrier comprises at least one belt and/or wire drive member which is driven by both motors.

8. Apparatus as claimed in claim 1 in which the feature comprises a pattern or symbol displayed on a liquid crystal display and the means for changing location is provided by suitable programming.

9. Apparatus as claimed in claim 2 including a light guiding member in which the at least two sources are arranged to inject light along an edge of said guiding member and the background comprises light emitted from a face of said guiding member.

10. Apparatus as claimed in claim 1 comprising an enclosure with an aperture through which, in use, the feature is observed by the subject and at least one face which comprises a surface for providing the illuminant.

11. Apparatus as claimed in claim 1 in which the feature comprises a dot.

12. Apparatus as claimed in claim 1 in which the feature comprises a pattern or symbol.

13. Apparatus as claimed in claim 1 in which the feature comprises an object.

14. Apparatus as Claimed in claim 13 in which the object is a ball.

15. Apparatus as claimed in claim 1 including computing means for controlling the tint and brightness of the illuminant and the location of the feature.

16. Apparatus as claimed in claim 1 which provides the subject or a practitioner with the means for controlling at least one of the tint of the background, the position of the feature and the motion of the feature.

17. A method for assessing and/or improving a subject's visual performance comprising presenting the subject with an observable feature against a background; changing the location of said feature along at least one locus of points; providing an illuminant to illuminate at least said background; and controlling the tint of the illuminant, wherein the steps of changing location and controlling tint are carried out within one assessment or improvement procedure.

18. A method for the simulation of the use of a filter by a subject under expected lighting conditions comprising:
defining the tristimulus values of a tint which would be observed under the expected lighting conditions by the subject when said filter is used in transmission for viewing an observable feature against a background;
providing a colour controllable source of light including narrowband coloured light sources;
presenting said feature against said background;
changing the location of said feature along at least one locus of points;
providing an illuminant to illuminate at least said background;
controlling the tint and level of the illuminant, to illuminate the background for viewing by the subject wherein the steps of changing location and controlling tint are carried out within one assessment or improvement procedure.

19. The method of claim 18 further comprising the step of simulating a range of pre-formulated filters and lighting conditions, whereby the subject can select one or more of said pre-formulated filters for use under said lighting conditions.

20. The method of claim 18 which includes the further step of formulating and/or selecting the filter to improve the subject's performance.

21. The method of claim 19 which includes the further step of formulating and/or selecting one of said preformulated filters to improve the subject's performance.

22. A method as claimed in claim 18 applied to the formulation of any one of filters and anti-reflection coatings for spectacles, contact lenses, coloured overlays and any other tinted material through which the subject may view the background and a purpose of which is to improve the subject's visual performance and/or stability.

23. An article formulated by the method of claim 22.
